(19) United States
(12) Patent Application Publication
(10) Pub. No.: US 2009/0226988 A1
(43) Pub. Date: Sep. 10, 2009

(54) Method for producing polymer

(75) Inventors: Kenji Tajima, Sapporo-shi (JP); Yasuharu Satou, Sapporo-shi (JP); Masanobu Munekata, Sapporo-shi (JP); Seiichi Taguchi, Sapporo-shi (JP); Tokuo Matsushima, Sapporo-shi (JP)

Correspondence Address: Quinn Emanuel Urquhart Oliver & Hedges, LLP;Koda/Androlia, 10th Floor, 865 S. Figueroa Street, Los Angeles, CA, 90017, US

(73) Assignees: National University Corporation Hokkaido University; Agribioindustry Inc.

(21) Appl. No.: 12/291,779

(22) Filed: Nov. 13, 2008

(30) Foreign Application Priority Data
Nov. 14, 2007 (JP) 2007-295145
Apr. 23, 2008 (JP) 2008-113203

(51) Int. Cl. C12P 7/62; C08G 63/02

(52) U.S. Cl. 435/135; 528/272

(57) ABSTRACT

A method for producing a polymer including a chemical thioester exchange reaction for forming an acetyl-CoA by reacting an acetyl-thioester with CoA, a monomer-producing reaction for forming a (monomer precursor)-CoA derivative by reacting at least one monomer precursor compound with the acetyl-CoA and a polymerization reaction for forming the polymer comprising units of the monomer by polymerizing the (monomer precursor)-CoA derivative.

### TECHNICAL FIELD

The present invention generally relates to a method for producing a polymer, and more particularly to a method for producing a biodegradable polymer for industrial application via a monomer-producing process by producing acetyl-coenzyme A (acetyl-CoA) using a acetate as a low-cost and starting substance, rather than a purified enzyme or expensive ATP.

### BACKGROUND ART

Among some polymers, chemical synthetic plastics derived from fossil fuels (e.g. petroleum), are unable to be degraded in natural environment and accumulate semipermanently in natural environment, resulting in various environmental problems. Under the circumstances, much attention has been focused on biodegradable plastics that are degraded by naturally-existing microorganisms (known as an eco-friendly polymeric material), and such material is increasingly developed so as to be provided with excellent properties towards practical use. In addition, the biodegradable plastics are expected to become leading biomaterials in biological and medical fields.

It has been conventionally known that biodegradable polyesters, which are produced and accumulated in many types of microorganisms from reproducible biological organic resources (biomass) such as sugar and vegetable oil. Among other things, polyhydroxyalkanoate (PHA) is expected to be an useful polyester due to thermoplasticity as the chemical synthetic plastics, excellent biodegradable and biocompatible properties, in which 90 or more-types of monomer structures have been found (see FEMS Microbiol. Lett., 1995, 128, pp. 219-228).

PHA is produced in a microorganism using fermentation thereby (in vivo synthetic method), or outside a microorganism using a purified PHA synthase (PHA synthetic enzyme) and a PHA's monomer compound (in vitro synthetic method). Currently, PHA is usually produced according to the in vivo synthetic method, but unfortunately the microbial fermentation technique can provide a limited production volume of PHA that merely accumulates in the microorganism and a high-cost process for pulverizing microorganisms to extract and purify PHA therefrom. In addition, the in vivo synthetic method fails to assuredly produce PHA having desired properties, and providing limited types of PHA synthesized due to complex microbial metabolic pathways. Some fermentation control methods may produce copolymers, rather than intended homopolymers, and even resulting copolymers could be non-uniform in desired molar ratio (see FEMS Microbiol. Rev., 1992, 103, pp. 207-214).

Currently, to solve the problems mentioned above, PHA production is increased by promoting PHA synthase expression or the composition of copolymerized PHA is controlled by converting substrate specificity {see Japanese Unexamined Patent Application Publications No. 1995-265065 and No. 1998-108682, and Japanese Unexamined Patent Application Publication (Translation of PCT Application) No. 2001-516574T}.

Meanwhile, the in vitro synthetic method was developed as mass-preparation of PHA synthase was achieved using recombinant DNA techniques (see Proc. Natl. Acad. Sci., 1995, 92, pp. 6279-6283), and thus it produces PHA by using a purified PHA synthase and monomer as a substrate as shown above. Accordingly, a monomer can be chemically prepared to extend PHA monomer structure and PHA production volume can be controlled with a high precision, thereby solving the problems in the in vivo synthetic method. The use of the in vitro synthetic method may produce PHA having various physical and functional properties that the in vivo synthetic method cannot obtain.

However, PHA production according to the in vitro synthetic method involves using an extremely expensive hydroxyacyl CoA (HA-CoA) as a substrate monomer, which must be continuously supplied. More disadvantageously, HA-CoA synthesis with an expensive CoA is significantly complex.

To overcome these problems, CoA that is released in the reaction system as PHA polymerization proceeds is reused, and HA-CoA is continuously supplied, with reference to, e.g., a document (FEMS Microbiology Letters, 1998, 168, pp. 319-324) disclosing a method for reusing CoA by forming an acetyl-CoA from CoA that is released with an acetate, an acetyl-CoA synthetase and ATP mixed together, and by forming 3-hydroxybutyl CoA with a propionyl-CoA transferase and a 3-hydroxybutyrate mixed together, and by forming poly-3-hydroxybutyrate from which 3-hydroxybutyryl CoA is polymerized (see FEMS Microbiology Letters, 1998, 168, pp. 319-324).

The document in WO2004065609 discloses a method for continuously supplying HA-CoA by reproducing CoA in PHA production process from thioester as a starting substance. More specifically, the thioester as a starting substance is reacted with CoA to form HA-CoA in a thioester exchange reaction, resulting in PHA production via polymerization by PHA synthase. In this process, CoA, which is released during this polymerization reaction, will be reproduced in an ester exchange reaction to continuously produce and reproduce HA-CoA.

### DISCLOSURE OF THE INVENTION

However, the method for producing PHA as disclosed in the document (FEMS Microbiology Letters, 1998, 168, pp. 319-324) involves using 3 types of enzymes that are difficult to be purified and extremely expensive ATP, leading to unachievable industrial application.

On the other hand, the invention disclosed in WO2004065609 produces a thiophenyl ester as a starting substance by thiophenyl-esterifying hydroxyalkanoate (HA). In reality, this thiophenyl esterification reaction cannot achieve CoA reproduction on industrial level, because this reaction requires a long-term process for the protection of HA's hydroxyl group and deprotection after the reaction.

In addition, according to the invention disclosed in WO2004065609, the thioester exchange reaction releases thiophenol as a remarkably toxic substance when acyl-CoA is formed from thiophenyl ester. Unfortunately, as polymerization reaction proceeds, several percents of thiophenol dissolve in an aqueous phase solution to inhibit the activity of PHA synthase.

It is, therefore, one object of the present invention to provide a method for producing a polymer of high production efficiency, productivity and thus industrial application, and a polymer composed of desired monomer units continuously producing and reproducing acetyl-CoA without using a purified enzyme or expensive ATP.

A method for producing a polymer according to the present invention comprises the following reaction, that is:

(a) a chemical thioester exchange reaction, wherein an acetyl-thioester is reacted with CoA for forming acetyl-CoA;

(b) a monomer-producing reaction, wherein at least one monomer precursor compound is reacted with the acetyl-CoA for forming a (monomer precursor)-CoA derivative;

(c) a polymerization reaction which the (monomer precursor)-CoA derivative is polymerized for forming the polymer comprising units of the monomer.

In this invention, the monomer precursor compound is a carboxylic acid and preferably a hydroxy acid.

Also, in this invention, polyester comprises units selected from serine or hydroxyalkanoates, preferably lactate (LA), 3-hydroxypropionate (3HP), 3-hydroxybutyrate (3HB), or 4-hydroxybutyrate (4HB), alone or in combination with each other or with other units.

In this invention, the polyester, preferably polylactate (PLA), poly-3-hydroxypropionate {P(3HP)}, poly-3-hydroxybutyrate {P(3HB)}, poly-4-hydroxybutyrate {P(4HB)}, LA-3HP-copolyester {P(LA-co-3HP)}, LA-3HB-copolyester {P(LA-co-3HB)}, LA-4HB-copolyester {P(LA-co-4HB)}, 3HP-3HB-copolyester {P(3HP-co-3HB)}, 3HP-4HB-copolyester {P(3HP-co-4HB)}, or 3HB-4HB-copolyester {P(3HB-co-4HB)} is produced as said polymer.

In this invention, the lactate (LA) is preferably Dextro-rotatory-lactate (D-lactate).

Moreover, in this invention, the chemical thioester exchange reaction preferably comprises forming acetyl-CoA from CoA, which is released from the (monomer precursor)-CoA derivative in the polymerization reaction, and the acetyl-thioester.

Also, it is preferable that in this invention, the acetyl-thioester of the chemical thioester exchange reaction is prepared from an acetate and a thiol compound by a thioesterification reaction.

In this invention, the thioesterification reaction comprises forming acetyl-thioester from acetate, which is released in the monomer-producing reaction, and thiol compounds.

Also in this invention, the thiol compound is preferably ethylthioglycolate (ETG).

Moreover, in this invention, an enzyme used for the monomer-producing reaction is one using acetyl-CoA as a substrate.

In this invention, it is preferable that the chemical thioester exchange reaction, the monomer-producing reaction and the polymerization reaction proceed concurrently in an one pot reaction system, the one pot reaction system preferably comprising an organic solvent phase and an aqueous solvent phase.

Also in this invention, the organic solvent phase comprises the acetyl-thioester, and the aqueous phase comprises CoA, the monomer precursor compound and two enzymes catalyzing the monomer-producing reaction (b) and the polymerization reaction (c), respectively.

It is desirable that in this invention, a ratio of the concentration of the acetyl-thioester and the concentration of the monomer precursor compound is 1:1 to 10:1.

Moreover, the method of the present invention provides for a targeted setting of the molecular weight distribution (Mw/Mn, wherein Mw denotes the weight-average molecular weight and Mn denotes the number-average molecular weight) of the polymer. The particular advantage of the present invention is that a polymer having a narrow molecular weight distribution, preferably between 1 and 3, and more preferably between 1 and 2.5 is provided.

Accordingly, it is, of course, that this invention can produce a polymer of a favorable industrial application in a high-yield, immediate and easy manner by continuously producing and reproducing acetyl-CoA without using a purified enzyme or expensive ATP. Also, this invention can produce a polymer with a desired polymer composition due to wider choices on monomer units.

### BRIEF DESCRIPTION OF THE DRAWINGS

The above and other objects of the invention will be seen by reference to the description taken in connection with the drawings, in which:

FIG. 1 is a diagram showing the reaction pathway for producing a polymer after forming acetyl-CoA from acetyl-thioester and forming (monomer precursor)-CoA derivative;

FIG. 2 is a diagram showing the reaction pathway for producing a polymer after forming acetyl-CoA from acetyl-thioester and forming (monomer precursor)-CoA derivative in the one pot reaction system;

FIG. 3 is a diagram showing the thioesterification reaction for forming acetyl-thioester from acetate;

FIG. 4 is a diagram showing the reaction pathway for producing P (3HB) after forming acetyl-CoA from acetyl-ETG and forming (monomer precursor)-CoA derivative in the one pot reaction system;

FIG. 5 is a diagram showing the reaction pathway for producing P (3HB-co-LA) after forming acetyl-CoA from acetyl-ETG and forming (monomer precursor)-CoA derivative in the one pot reaction system;

FIG. 6 is a diagram showing the reaction (1) for forming acetyl-TP from acetate in Example 1 and the reaction (2) for forming acetyl-ETG from acetate in Example 1;

FIG. 7 is a diagram showing the reaction pathway (1) for producing P (3HB) after forming acetyl-CoA from acetyl-TP and forming (R)-3HB-CoA and the reaction pathway (2) for producing P (3HB) after forming acetyl-CoA from acetyl-ETG and forming (R)-3HB-CoA (“n” in FIG. 7 is an integer 1 or more);

FIG. 8 is a graph showing the results of NMR measured for compounds obtained using acetyl-ETG with the same concentration as monomer precursor compound in Example 1;

FIG. 9 is a graph showing the results of NMR measured for compounds obtained using acetyl-ETG with the concentration 10 times that of monomer precursor compound in Example 1;

FIG. 10 is a graph showing the results of synthetic reaction rate and synthetic amount measured in P (3HB according to the type of acetyl-thioester in Example 2;

FIG. 11 is a graph showing the results of reaction rate and amount measured in P (3HB) production according to difference in the ratio of the volume of the organic solvent phase comprising acetyl-ETG and the amount of the aqueous phase comprising (R)-3HB in Example 3;

FIG. 12 is a diagram showing the reaction pathway for producing P (3HB-co-3HP) after forming acetyl-CoA from acetyl-ETG and forming (R)-3HB-CoA or 3HP-CoA in Example 4 (“x” and “y” in FIG. 12 are integers 1 or more); and

FIG. 13 is a graph showing the results of NMR measured for compounds obtained in Example 4.

FIG. 14 is a diagram showing the reaction pathway for producing P (3HB-co-LA) after forming acetyl-CoA from acetyl-ETG and forming (R)-3HB-CoA or LA-CoA in Example 5 (“x” and “y” in FIG. 14 are integers 1 or more); and

FIG. 15 is a graph showing the results of NMR measured for compounds obtained in Example 5.

### BEST MODE FOR CARRYING OUT THE INVENTION

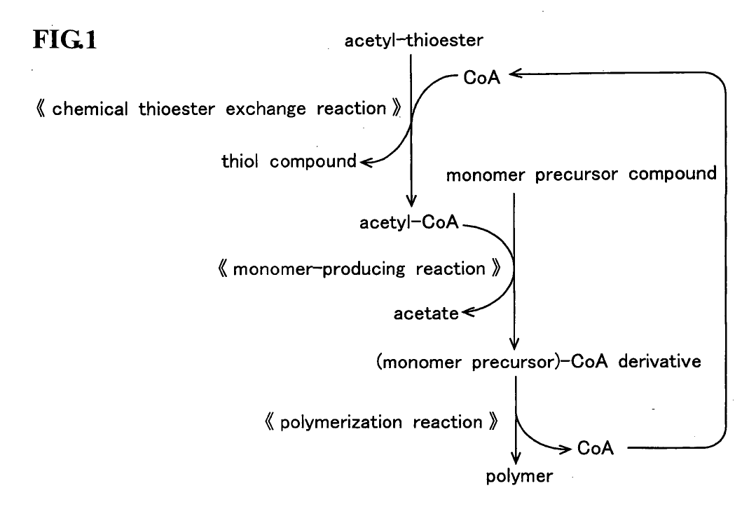

A preferred embodiment of the method for producing a polymer according to the present invention will be described with reference to FIGS. 1 to 3. As shown in FIG. 1, the method for producing a polymer of this embodiment comprises a chemical thioester exchange reaction, a monomer-producing reaction and a polymerization reaction.

The chemical thioester exchange reaction is a chemical reaction that reacts an acetyl-thioester as an acetyl donor with CoA to form acetyl-CoA and releases thiol compounds. The monomer-producing reaction reacts the acetyl-CoA produced in the chemical thioester exchange reaction with at least one monomer precursor compound, forms a (monomer precursor)-CoA derivative by substituting CoA for a hydroxyl group of the monomer precursor compound and releases acetate. The polymerization reaction forms a polymer by polymerizing the (monomer precursor)-CoA derivative obtained by the monomer-producing reaction and releases CoA.

A monomer precursor compound in the monomer-producing reaction may be carboxylic acid, preferably hydroxy acid or unsaturated fatty acid. The hydroxy acid or unsaturated fatty acid is not particularly limited if it forms a (monomer precursor)-CoA derivative by the monomer-producing reaction and then produces a polymer by the polymerization reaction after, but may be aliphatic hydroxy acid such as tartrate, glycerate, acrylate, crotonate, aminocrotonate, hydroxycrotonate, pentenate, hexanoate, octanoate, malic acid, tartaric acid, citramalic acid, citric acid, isocitric acid, leucic acid, mevalonic acid, pantoic acid, ricinoleic acid, ricinelaidic acid, cerebronic acid, quinic acid, shikimic acid, serine (Ser) and HA, or aromatic hydroxy acid such as salicylic acid, hydroxymethyl benzoic acid, vanillic acid, syringic acid, pyrocatechuic acid, resorcyclic acid, protocatechuic acid, gentisic acid, orsellinic acid, gallic acid, mandelic acid, benzilic acid, atrolactic acid, melilotic acid, phloretic acid, coumaric acid, umbellic acid, caffeic acid, ferulic acid and sinapic acid, preferably Ser or HA.

Next, HA in this invention may be lactic acid (2-hydroxypropionate; LA), glycolic acid, 3-hydroxypropionate (3HP), 3-hydroxybutyrate (3HB), 3-hydroxy valerate (3HV), 3-hydroxyhexanoate, 3-hydroxyheptanoate, 3-hydroxyoctanoate, 3-hydroxynonanoate, 3-hydroxydecanoate, 3-hydroxyundecanoate, 3-hydroxydodecanoate, 3-hydroxydodecenoate, 3-hydroxytetradecanoate, 3-hydroxyhexadecanoate, 3-hydroxyoctadecanoate, 4-hydroxybutyrate (4HB), 4-hydroxy valerate, 5-hydroxy valerate, 6-hydroxyhexanoate and hydroxylauric acid, preferably LA, 3HP, 3HB or 4HB.

There are D-enantiomer and L-enantiomer of LA. LA as a monomer precursor compound in this invention may be both of them, preferably D-enantiomer in this embodiment.

The polymers that can be produced in the method for producing a polymer according to this invention include polyesters. The polyesters in this invention may be PHA that homopolymers of the HA or copolymers, randompolymers or blockpolymers comprising the HA selected from the HA, preferably poly LA (PLA), poly (3-hydroxypropionate) {P(3HP)}, poly(3-hydroxybutyrate) {P(3HB)}, poly (4-hydroxybutyrate) {P(4HB)}, copolymer of LA and 3HP {P(LA-co-3HP)}, copolymer of LA and 3HB {P(LA-co-3HB)}, copolymer of LA and 4HB {P (LA-co-4HB)}, copolymer of 3HP and 3HB {P(3HP-co-3HB)}, copolymer of 3HP and 4HB {P(3HP-co-4HB)} and copolymer of 3HB and 4HB {P(3HB-co-4HB)}. In addition, the polyesters include units selected from Ser or HA. Therefore these units may be preferable that homopolymer of Ser (PS), copolymers, randompolymers or blockpolymers comprising Ser and HA selected from 3HP, 3HB or 4HB, alone, or in combination with each other or with other units. It is preferable that the polymers in this invention are biodegradable.

The method for producing a polymer according to the present invention can form an acetyl-CoA by reacting CoA released in the polymerization reaction with an acetyl-thioester in the chemical thioester exchange reaction. This means, CoA used in forming the acetyl-CoA in the chemical thioester exchange reaction can be reproduced from CoA released from a (monomer precursor)-CoA derivative in the polymerization reaction. Also, CoA can be supplied to form acetyl-CoA in the chemical thioester exchange reaction.

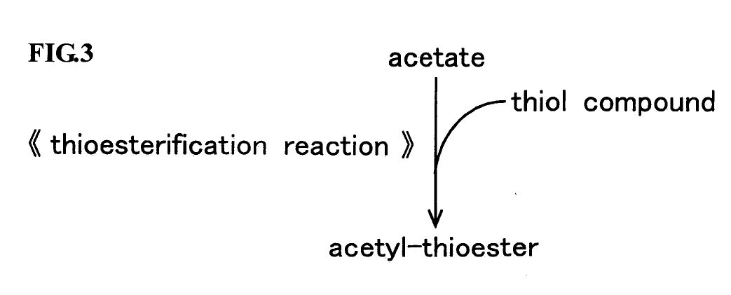

A method for producing acetyl-thioester as a starting substance in the chemical thioester exchange reaction is not particularly limited if acetyl-thioester can be formed using low-cost compounds, but preferably employs a thioesterification reaction for reacting acetate with thiol compounds, as shown in FIG. 3, in view of one-step and easy process without using expensive compounds.

Meanwhile, acetate released in the monomer-producing reaction reacts with thiol compounds in the thioesterification reaction, resulting in the production of acetyl-thioester. Specifically, acetate used in forming acetyl-thioester in the thioesterification reaction can be reproduced from acetate released in the monomer-producing reaction. Also, acetate can be supplied to form acetyl-thioester in the thioesterification reaction.

Here, thiol compounds in this invention are not particularly limited if they have a thiol group that can be given to CoA to form an acetyl-CoA, but preferably aliphatic thiol and aromatic thiol, and more specifically alkylthiol and cycloalkylthiol of C1 to C 18 such as ethanethiol, propanethiol, benzylmercaptan, 2-mercaptoethyl ether and cyclohexylthiol, thiol comprising hydroxyl groups such as mercaptoethanol and p-mercaptophenol, thiol comprising carboxylic acid ester such as methylmercaptoacetate and ethylmercaptopropionate, aromatic thiol such as thiophenol (TP), toluenethiol and naphthalenethiol, nitrogenous aromatic thiol such as 2-mercapto-1-methylimidazole, mercaptopyridine, mercaptothiazoline, mercaptobenzothiazoline, mercaptobenzoxazole and mercaptopyrimidine, thioglycolate such as methylthioglycolate, ethylthioglycolate (ETG), propylthioglycolate, isopropylthioglycolate, butylthioglycolate, n-amylthioglycolate, isoamylthioglycolate, hexylthioglycolate, octylthioglycolate, n-decylthioglycolate, laurylthioglycolate, tridecylthioglycolate, stearylthioglycolate, thionalide, furfurylmercaptan, cyclohexylthioglycolate and hydroxyethylthioglycolate, preferably TP or thioglycolate, more preferably thioglycolate. A preferred thiol compound in this embodiment is ETG due to an ability to form acetyl-thioester from acetate stably, maintain synthase activity for untoxic property, and high production rate and high production yield of polymer.

On the other hand, an enzyme used in the monomer-producing reaction is preferably one using acetyl-CoA as a substrate, because the monomer-producing reaction forms a (monomer precursor)-CoA derivative from acetyl-CoA and the hydroxy acid as a substrate. The enzyme having acetyl-CoA as a substrate may be CoA transferase. The CoA transferase may be acetoacetyl-CoA transferase, caffeoyl-CoA transferase, coumaroyl-CoA transferase, glutaryl-CoA transferase, crotonyl-CoA transferase, sinapoyl-CoA transferase, cinnamoyl-CoA transferase, succinyl-CoA transferase, 3-hydroxybutanoyl-CoA transferase, hydroxymethyl glutaryl-CoA transferase, feruloyl-CoA transferase, propionyl-CoA transferase (PCT) and malonyl-CoA transferase, furthermore a favorable CoA transferase can be selected according to hydroxy acid as a substrate. For example, if hydroxy acid is 3HB, 3HP, 4HB, LA or Ser, PCT can be used.

The CoA transferase-derived microorganisms are not particularly limited if they have an enzyme that can transfer a CoA group from the acetyl-CoA to the monomer precursor compound, but if the enzyme is propionyl-CoA transferase, the microorganisms may be genus Clostridium, genus Megasphaera, genus Alkaliphilus, genus Thermosinus, genus Pelotomaculum, genus Listeria, genus Ralstonia, genus Syntrophobacter, genus Fusobacterium, genus Syntrophus, genus Mycobacterium, genus Dechloromonas, genus Bacillus, genus Rhodoferax, genus Bradyrhizobium, genus Polynucleobacter, genus Eubacterium, genus Rhizobium, genus Oceanospirillum, genus Vibrio, genus Burkholderia, genus Pseudomonas, genus Shewanella and genus Escherichia, more specifically Clostridium propionicum, Clostridium kluyveri, Megaspkaera elsdenii, Clostridium novyi, Clostridium tetani, Clostridium perfringens, Clostridium beijerinckii, Alkaliphilus metalliredigens, Alkaliphilus oremlandii, Thermosinus carboxydivorans, Pelotomaculum thermopropionicum, Listeria monocytogenes, Listeria welshimeri, Ralstonia eutropha, Syntrophobacter fumaroxidans, Fusobacterium nucleatum, Syntrophus aciditrophicus, Mycobacterium smegmatis, Dechloromonas aromatica, Bacillus halodurans, Rhodoferax ferrireducens, Bradyrhizobium japonicum, Polynucleobacter sp., Eubacterium dolichum, Rhizobium leguminosarum, Oceanospirillum sp., Vibrio shilonii, Burkholderia phytofirmans, Pseudomonas mendocina, Shewanella sediminis and Escherichia coli.

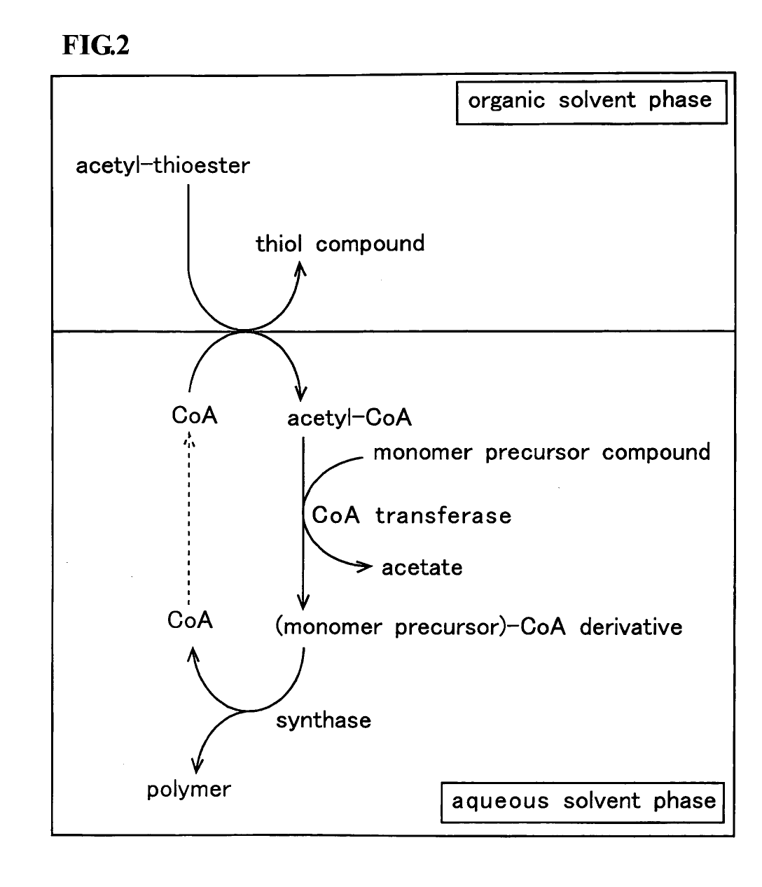

Next, the method for producing a polymer according to the present invention includes at least the chemical thioester exchange reaction, the monomer-producing reaction and the polymerization reaction, and may also include other reactions like hydration by hydratase. In addition, the chemical thioester exchange reaction, the monomer-producing reaction and the polymerization reaction can separately proceed, and as shown in FIG. 2, they can concurrently proceed in one pot reaction system. The one pot reaction system can include the thioesterification reaction, and preferably comprises an organic solvent phase and an aqueous phase.

To produce a polymer in the one pot reaction system including the organic solvent phase and the aqueous phase, it is preferable that the organic solvent phase comprises acetyl-thioester, and the aqueous phase includes CoA, monomer precursor compound CoA transferase and synthase. As shown in FIG. 2, the acetyl-thioester in the organic solvent phase and CoA in the aqueous phase form thiol compounds and acetyl-CoA by the chemical thioester exchange reaction at the interface therebetween, and acetyl-CoA and monomer precursor compound form acetate and (monomer precursor)-CoA derivative in the aqueous phase by the monomer-producing reaction by catalysis of an enzyme using acetyl-CoA as a substrate. Subsequently, (monomer precursor)-CoA derivatives in the aqueous phase are polymerized to produce a polymer by catalysis of an enzyme according to the polymerization reaction.

Now, the enzyme according to the polymerization reaction may be PHA synthase. PHA synthase in this invention includes enzymes that polymerize not only HA for forming PHA, but also LA for forming PHA, and HA and LA for forming copolymers, randompolymers or blockpolymers. PHA synthase is favourably selected according to the (monomer precursor)-CoA derivative as a substrate. For instance, when the (monomer precursor)-CoA derivative is 3HB-CoA, 3HP-CoA, 4HB-CoA or LA-CoA, alone or in combination with each other or with other units, PHA synthase of genus Ralstonia-derived, genus Pseudomonas-derived, or conservative-amino-acid replacement variant-derived can be used.

The PHA synthase-derived microorganisms are not particularly limited if they have an enzyme that can synthesize PHA using HA-CoA as a substrate, but the microorganisms may be genus Ralstonia, genus Burkholderia, genus Methylibium, genus Pseudomonas, genus Cupriavidus, genus Polaromonas, genus Alcaligenes, genus Azohydromonas, genus Rhodoferax, genus Acidovorax, genus Verminephrobacter, genus Polynucleobacter, genus Bordetella, genus Zoogloea, genus Herminiimonas, genus Dechloromonas, genus Azoarcus, genus Bradyrhizobium, genus Azotobacter, genus Oceanospirillum, genus Chromobacterium, genus Nitrococcus, genus Alkalilimnicola, genus Magnetospirillum, genus Halorhodospira, genus Rhodospirillum, genus Rubrobacter, genus Parvibaculum, genus Acidiphilium, genus Sphingomonas, genus Saccharophagus, genus Photobacterium, genus Chromohalobacter, genus Azorhizobium, genus Methylobacterium, genus Vibrio, genus Rhodopseudomonas, genus Bacillus, genus Roseiflexus, genus Syntrophomonas, genus Chloroflexus, genus Myxococcus, genus Novosphingobium, genus Haloarcula, genus Cenarchaeum, genus Synechococcus, genus Synechocystis, genus Allochromatium, genus Microscilla, genus Chlorogloeopsis, genus Ectothiorhodospira, genus Xanthomonas, genus Nitrococcus mobilis, genus Marinobacter, genus Alcanivorax, genus Hahella, genus Acinetobacter, genus Aeromonas, genus Limnobacter and genus Parvularcula, and more specifically Ralstonia eutropha, Ralstonia metallidurans, Ralstonia solanacearum, Ralstonia pickettii, Burkholderia multivorans, Burkholderia pseudomallei, Burkholderia dolosa, Burkholderia mallei, Burkholderia ambifaria, Burkholderia cenocepacia, Burkholderia thailandensis, Burkholderia phymatum, Burkholderia xenovorans, Burkholderia vietnamiensis, Methylibium petroleiphilum, Pseudomonas putida, Pseudomonas oleovorans, Cupriavidus necator, Polaromonas naphthalenivorans, Alcaligenes sp., Azohydromonas lata, Rhodoferax ferrireducens, Acidovorax avenae, Verminephrobacter eiseniae, Polynucleobacter sp., Bordetella pertussis, Zoogloea ramigera, Bordetella bronchiseptica, Bordetella parapertussis, Bordetella avium, Herminiimonas arsenicoxydans, Limnobacter sp., Dechloromonas aromatica, Azoarcus sp., Bradyrhizobium japonicum, Azotobacter vinelandii, Oceanospirillum sp., Chromobacterium violaceum, Nitrococcus mobilis, Alkalilimnicola ehrlichei, Magnetospirillum magneticum, Halorhodospira halophila, Rhodospirillum rubrum, Magnetospirillum gryphiswaldense, Rubrobacter xylanophilus, Parvibaculum lavamentivorans, Acidiphilium cryptum, Sphingomonas sp., Saccharophagus degradans, Photobacterium profundum, Chromohalobacter salexigens, Azorhizobium caulinodans, Methylobacterium sp., Vibrio alginolyticus, Rhodopseudomonas palustris, Bacillus anthracis, Bacillus cereus, Bacillus thuringiensis, Bacillus weihenstephanensis, Bacillus megaterium, Rubrobacter xylanophilus, Roseiflexus castenholzii, Syntrophomonas wolfei, Chloroflexus aggregans, Myxococcus xanthus, Novosphingobium aromaticivorans, Haloarcula marismortui, Haloarcula hispanica, Halorhodospira halophila, Cenarchaeum symbiosum, Synechococcus sp., Synechocystis sp., Allochromatium vinosum, Microscilla marina, Chlorogloeopsis fritschii, Ectothiorhodospira shaposhnikovii, Xanthomonas campestris, Nitrococcus mobilis, Marinobacter aquaeolei, Alcanivorax borkumensis, Hahella chejuensis, Acinetobacter baumannii, Aeromonas salmonicida and Parvularcula bermudensis.

In the method for producing a polymer according to this invention, when the chemical thioester exchange reaction, the monomer-producing reaction, and the polymerization reaction concurrently proceed in the one pot reaction system, a ratio (mmol/L) of the molecular concentration of the acetyl-thioester in the organic solvent phase and the molecular concentration of the monomer precursor in the aqueous phase is preferably 1:1 to 10:1 in view of production rate and production yield.

The molecular weight distribution for a polymer produced in the method for producing a polymer according to this invention is not particularly limited if the polymer is produced by the above-mentioned method, but it is preferably between 1 and 3, and more preferably between 1 and 2.5 in view of the quality and repeatability in polymer production.

As stated above, the method for forming acetyl-CoA via forming acetyl-thioester from acetate in this embodiment is industrially useful due to low-cost substances such as acetate as a raw material, without using expensive materials such as ATP and enzymes requiring to be purified or carefully handled with. Additionally, a released monomer precursor compound, such as LA produced by readily produced and low-cost lactic acid fermentation, can be directly used in the monomer-producing reaction. Specifically, the method for producing a polymer according to this invention can readily produce a polymer having a desired composition by selecting a desired monomer.

Next, an embodiment of the method for producing a polymer according to the present invention will be described. In this embodiment, an acetyl-ETG is formed from acetate and ETG by a thioesterification reaction. By adding the acetyl-ETG to an organic solvent phase composed of hexane and adding CoA, HA, CoA transferase and PHA synthase to an aqueous phase, the chemical thioester exchange reaction, the monomer-producing reaction and the polymerization reaction gradually proceed to produce a polymer.

Specifically, the acetyl-ETG in a hexane phase and the CoA in an aqueous phase cause a chemical thioester exchange reaction at the interface therebetween to release ETG is released in the hexane phase and form acetyl-CoA in the aqueous phase. Subsequently, the acetyl-CoA and HA release acetate in the aqueous phase by a monomer-producing reaction by CoA transferase and form a HA-CoA derivative. Finally, the HA-CoA is polymerized by the PHA synthase in the aqueous phase to release CoA in the aqueous phase and to produce a polymer.

The CoA released in the aqueous phase in the polymerization reaction is reproduced in the chemical thioester exchange reaction, thereby continuously supplying acetyl-CoA because of CoA recycling. In addition, acetate that is released in the aqueous phase in the monomer-producing reaction can be extracted to reuse as a raw material for acetyl-ETG.

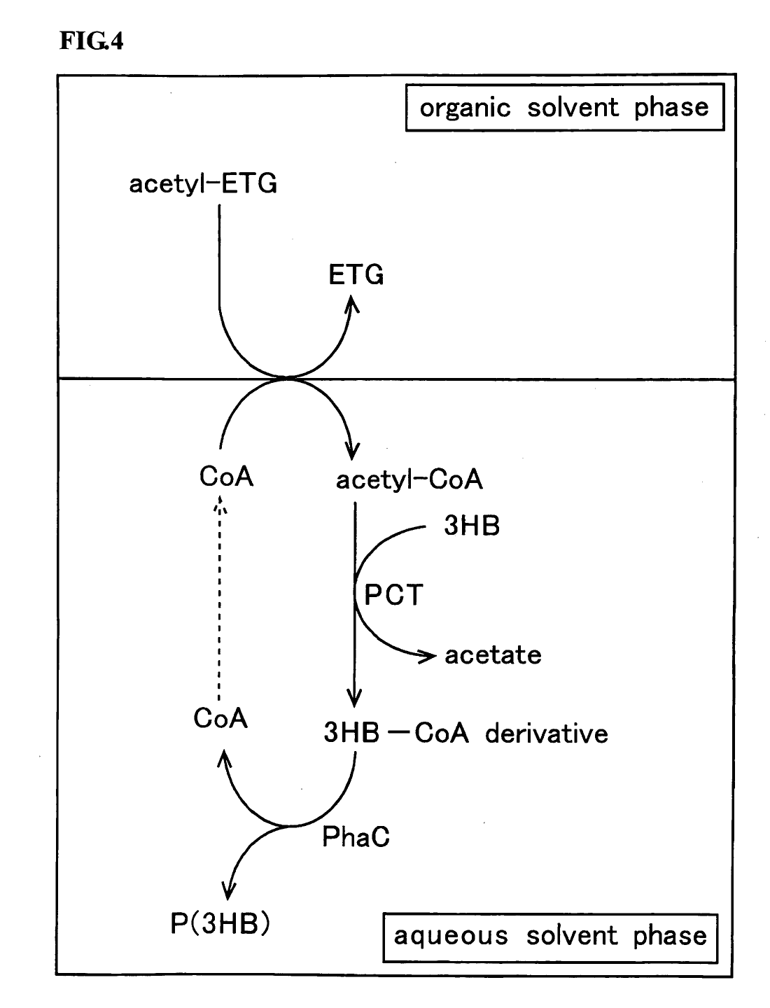

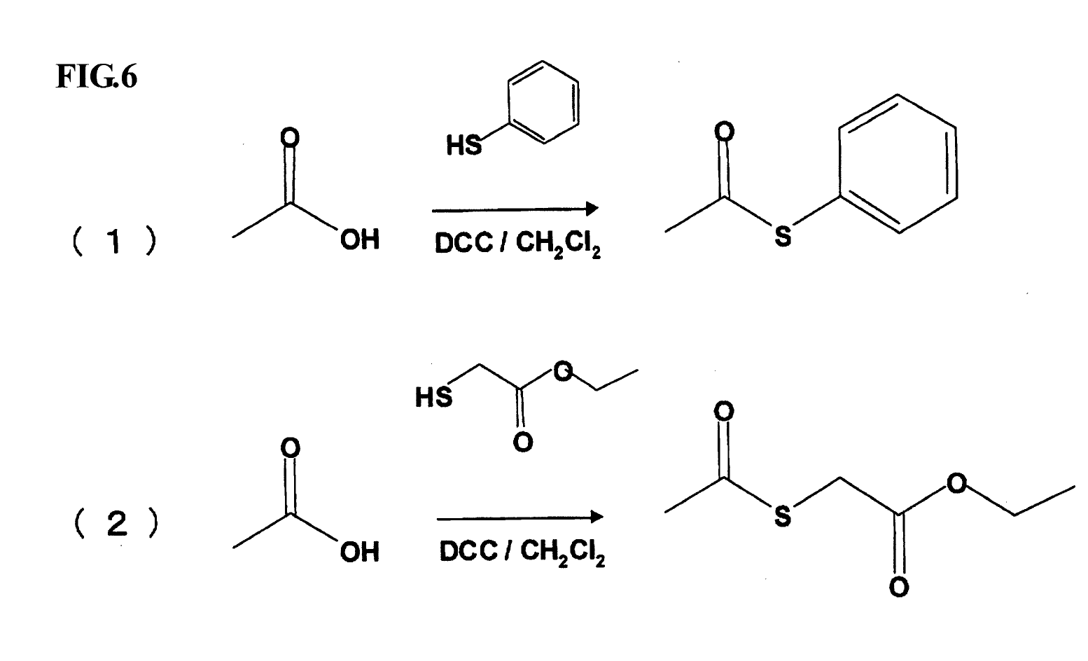

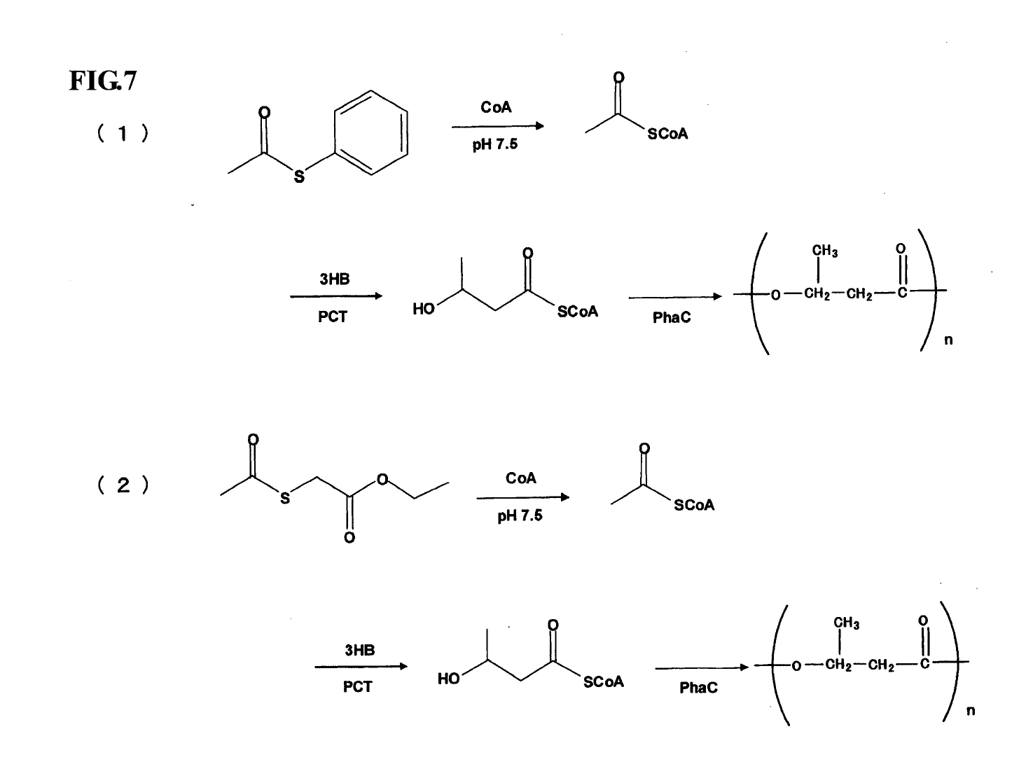

Then, in this embodiment, a method for forming P (3HB) after forming acetyl-CoA from acetate and ETG and forming 3HB-CoA derivative will be described with reference to FIG. 4, FIG. 6 (2) and FIG. 7 (2).

Firstly, a method for forming an acetyl-ETG from acetate in one step by a thioesterification reaction will be described. As shown in FIG. 6 (2), by reacting acetate with ETG in dichloromethane (CH₂Cl₂) having dicyclohexylcarbodiimide (DCC), acetyl-ETG is formed in one step.

Next, in this embodiment, a method for producing P (3HB) from acetyl-ETG and 3HB in an one pot reaction system will be described. Specifically, as shown in FIG. 4, the chemical thioester exchange reaction, the monomer-producing reaction for forming 3HB-CoA derivative from 3HB as a monomer component and the polymerization reaction for producing P (3HB) concurrently proceed in the one pot reaction system.

In this production method, by dissolving the acetyl-ETG in an organic solvent phase using hexane and adding CoA, 3HB, PCT and PHA synthase to a sodium hydrogenphosphate solution as an aqueous phase, a chemical thioester exchange reaction is performed at the interface between the organic solvent phase and the aqueous phase. Subsequently, by performing the monomer-producing reaction and the polymerization reaction in the aqueous phase, P (3HB) is produced. FIG. 7(2) shows the reaction pathway.

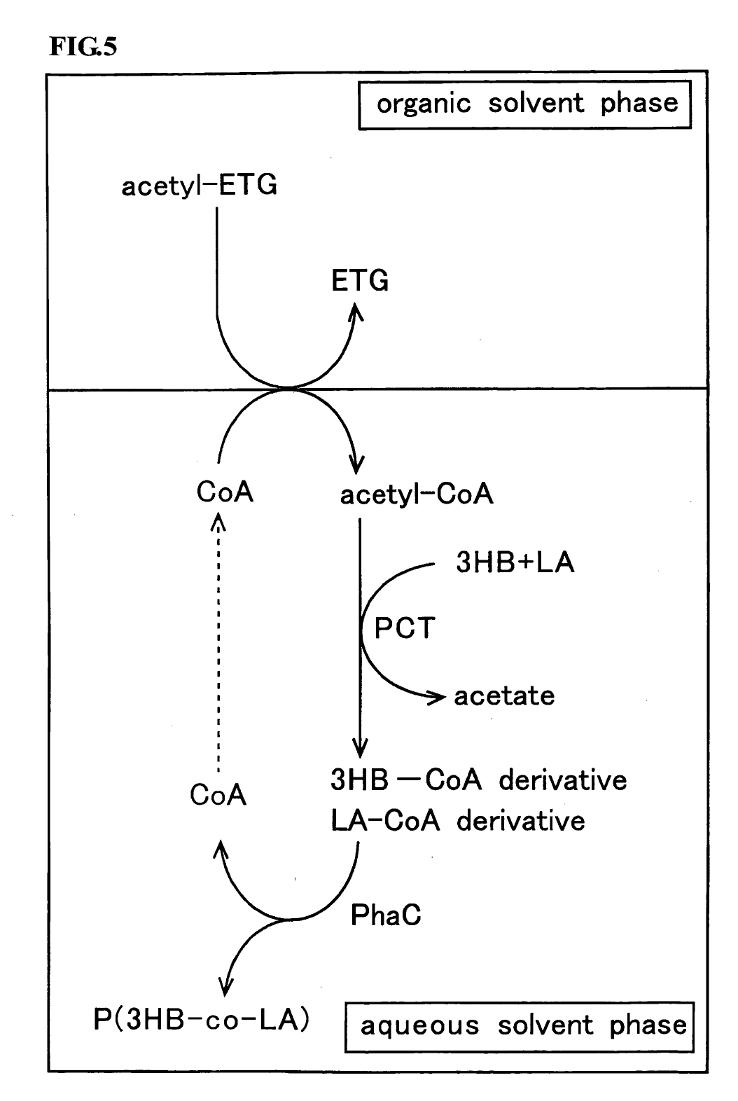

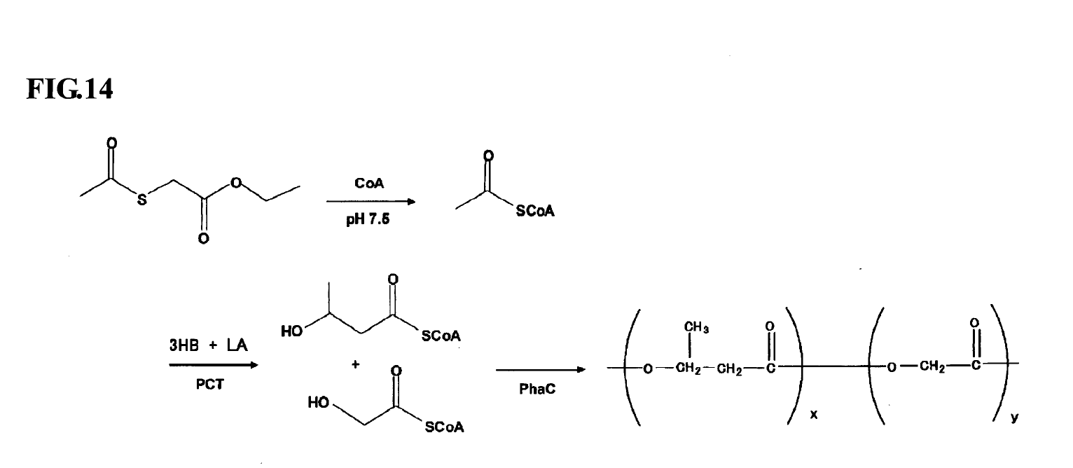

Subsequently, in this embodiment, a method for forming P (3HB-co-LA) after forming acetyl-CoA from acetate and ETG and forming 3HB-CoA derivative and 3LA-CoA derivative will be described with reference to FIG. 5, FIG. 6(2) and FIG. 14.

Firstly, a method for forming an acetyl-ETG from acetate in one step by a thioesterification reaction will be described. As shown in FIG. 6(2), by reacting acetate with ETG in dichloromethane (CH₂Cl₂) having dicyclohexylcarbodiimide (DCC), acetyl-ETG is formed in one step.

Next, in this embodiment, a method for producing P (3HB-co-LA) from acetyl-ETG, 3HB and LA in an one pot reaction system will be described. Specifically, as shown in FIG. 5, the chemical thioester exchange reaction, the monomer-producing reaction for forming 3HB-CoA derivative and LA-CoA derivative from 3HB and LA respectively as monomer components and the polymerization reaction for producing P (3HB-co-LA) concurrently proceed in the one pot reaction system.

In this production method, by dissolving the acetyl-ETG in an organic solvent phase using hexane and adding CoA, 3HB, LA, PCT and PHA synthase to a sodium hydrogenphosphate solution as an aqueous phase, a chemical thioester exchange reaction is performed at the interface between the organic solvent phase and the aqueous phase. Subsequently, by performing the monomer-producing reaction and the polymerization reaction in the aqueous phase, P (3HB-co-LA) is produced. FIG. 14 shows the reaction pathway.

In this embodiment, the ratio of the volumes in the organic solvent phase and the aqueous phase is not particularly limited if P (3HB), P (3HB-co-3HP) or P (3HB-co-LA) as an objective product can be produced from the acetyl-ETG in the organic solvent phase. However, in view of more immediate and high-yield production, a ratio (mmol/mL) of the molecular concentration of the acetyl-ETG in the organic solvent phase and the concentration of the monomer compounds in the aqueous phase is preferably 1:1 to 10:1.

In this embodiment, PCT is not particularly limited if it is derived from a microorganism having an enzyme that can transfer a CoA group from an acetyl-CoA to 3HP, 3HB or LA but it is Clostridium propionicum, particularly Clostridium propionicum JCM1430 in this embodiment.

Additionally, in this embodiment, PHA synthase is not particularly limited if it is derived from a microorganism having an enzyme that can polymerize 3HB-CoA derivatives, 3HP-CoA derivatives or LA-CoA derivatives, alone, or in combination with each other and synthesize P (3HB), P (3HB-co-3HP) or P (3HB-co-LA) but it is microorganism-derived that belongs to genus Ralstonia and genus Pseudomonas, particularly, Ralstonia eutropha, and Pseudomonas sp. 61-3. In addition, Ralstonia eutropha ATCC 17699-derived and Sequence No. 2 as a preferred PHA synthase in this embodiment.

### EXAMPLES

Next, specific examples in the method for producing a polymer of this embodiment are described.

### Example 1

In Example 1, a method for forming acetyl-thioester using acetate as a starting material to produce P (3HB) is described.

(1) Production of Acetyl-Thioester

First, using acetate as a raw material, 2 types of acetyl-thioester (acetyl-TP and acetyl-ETG) were prepared [Yuan, W.; Jia, Y.; Tian, J.; Snell, K. D.; Müh, U.; Sinskey, A. J.; Lambalot, R. H.; Walsh, C. T.; Stubbe, J. Arch. Biochem, Biophys. 2001, 394, 87-98.]. FIG. 6 (1) shows the production process for acetyl-TP and FIG. 6 (2) shows the production process for acetyl-ETG.

It was confirmed that each substance obtained is esterificated by thin-layer chromatography (TLC) technique, using instrument of Merck Ltd. (Silica Gel F254). The overall structure was found by ¹H-NMR spectrum, using nuclear magnetic resonance (NMR). In NMR measurement, MSL400 spectroscope of Brunker Corporation was employed, with a frequency of 400 MHz. All NMR spectra were recorded in deuterated chloroform (CDCl₃) as the solvent, wherein the Figures only show sections of the spectra within a range of 0 to about 6.4 ppm. In the range above 6.4 ppm, there were no relevant signals, therefore this range is omitted and the typical singlet signal of CDCl₃ at 7.24 ppm is not shown.

(2) PHA Synthase (PhaC)

Next, after microbial production of overexpression PhaC was constructed, purified PhaC was obtained (Satoh, Y.; Tajima, K.; Tannai, H.; Munekata, M. J. Biosci. Bioeng. 2003, 95, 335-341).

Firstly, Genomic DNA of Ralstonia eutropha ATCC 17699 was treated with restriction enzymes of EcoRI and SmaI (both prepared by TAKARA Bio Inc.). Using pUC18 (TAKARA Bio Inc.), approx. 5 kbp of gene fragment containing a PhaC gene was cloned to obtain a plasmid pTI305.

Next, approx. 1.6 kbp of NotI/Stul fragment in a pTI305, a gene fragment having 140 bp of BamHI site and SmaI site amplified by PCR according to the following conditions, using pTI305 as a template, and vector pQE 30 (Qiagen) treated with BamHI and SmaI were mixed to be ligated. Then, using this reaction solution, Escherichia coli JM109 was transformed to obtain a plasmid pQEREC having a PhaC gene from a transfectant. By introducing this plasmid to an Escherichia coli BL21, Escherichia coli for preparing PhaC was obtained.

The PCR employed the following primers.

Sense primer: aaggatccatggcgaccggcaaaggcgcgg (Sequence No. 3)

Antisense primer: tgcagcggaccggtggcctcggcc (Sequence No. 4)

The PCR was performed in 30 cycles, each cycle comprising 45-second reaction at 94° C., 30-second reaction at 58° C., and 60-second reaction at 72° C.

Escherichia coli for preparing PhaC obtained was cultured in 1000 mL of LB medium containing ampicillin at 30° C. for 16 hours. After sonication of the microbial cell bodies accumulated PhaC, soluble protein in the microbial cell body was collected. The collected protein was put in an Ni-NTA agarose gel column (Qiagen) to purify 6×His)-PhaC in one step.

(3) PCT

Next, using a transformant obtained by introducing a plasmid pCCPP to the Escherichia coli BL21, Clostridium propionicum-derived PCT was produced to obtain a purified PCT by the same approach as the PhaC purification.

The activity of the purified PCT was measured using the monomer-producing reaction and P (3HB) polymerization reaction combined. Specifically, 0.5 mL of solution containing 100 mM sodium phosphate buffer (pH7.5), 2 mM acetyl-CoA and 200 mM 3HB, PhaC and PCT was prepared. Then, PCT activity was confirmed by observing the rise in CoA concentration as P (3HB) was produced.

(4) Production of P (3HB)

Then, P (3HB) was produced, using the acetyl-TP or acetyl-ETG, and the PhaC, and the PCT obtained in the above processes. FIG. 7(1) shows the reaction process using the acetyl-TP, and FIG. 7(2) shows the reaction process using the acetyl-ETG.

Firstly, as an aqueous phase reaction solution, 5 mL of solution containing 100 mM sodium phosphate buffer (pH7.5), 2.0 mM CoA, 10 mM (R)-3HB and 25 U (1 mg) PCT was prepared. Next, as an organic solvent phase reaction solution, 5 mL of hexane solution containing 10 mM acetyl-TP or 10 mM acetyl-ETG was prepared. After pouring the aqueous phase reaction solution into a screw cap test tube, the organic solvent phase reaction solution was added thereto. Finally, 5.4 U (0.2 mg) PhaC was added to the aqueous phase to be reacted at 30° C. for 48 hours. The organic solvent phase was removed after the reaction was completed, and 5 mL of chloroform was added thereto to extract a product at 70° C. for 3 hours. The extract was filtrated with a filter (0.2 μm PTFE membrane; Advantec), and 50 mL of methanol was added thereto and allowed to stand overnight at 4° C. Afterwards, a produced precipitate was filtrated with a filter (0.2 μm PTFE membrane) and collected. After it was vacuum-dried, its yield was measured. 0.2 mg of an acetyl-TP product and 2.9 mg of an acetyl-ETG product were obtained.

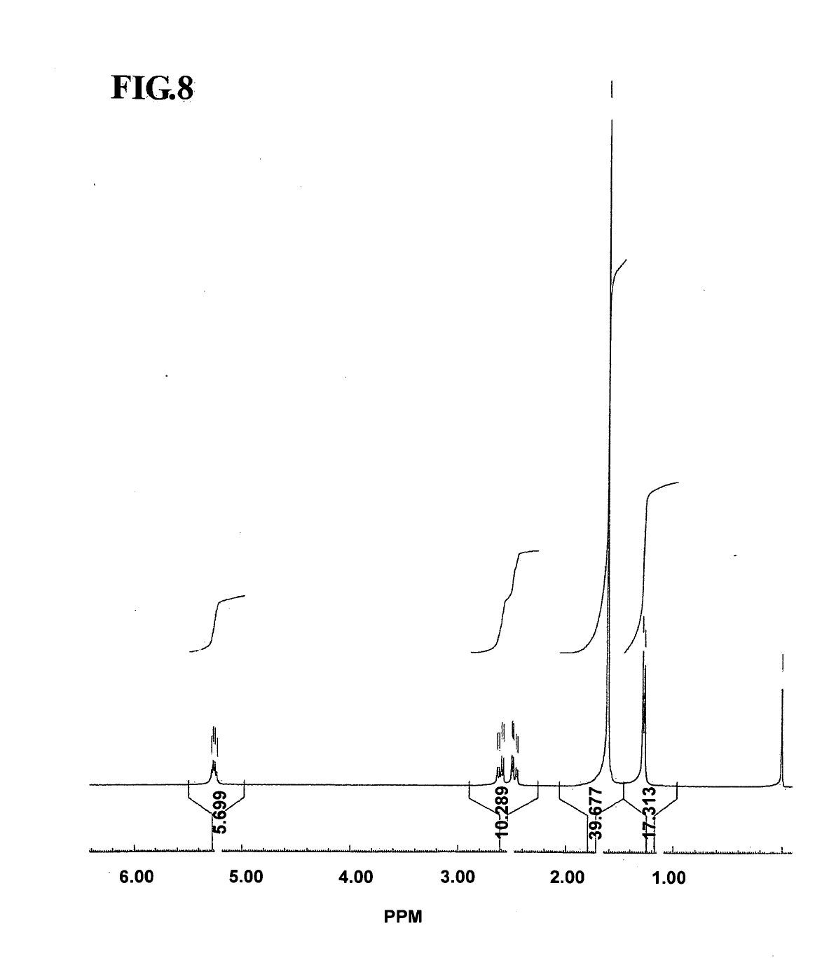

The structure of each product obtained was confirmed using NMR to find-out a P (3HB) product. FIG. 8 shows ¹H-NMR spectrum using the acetyl-ETG.

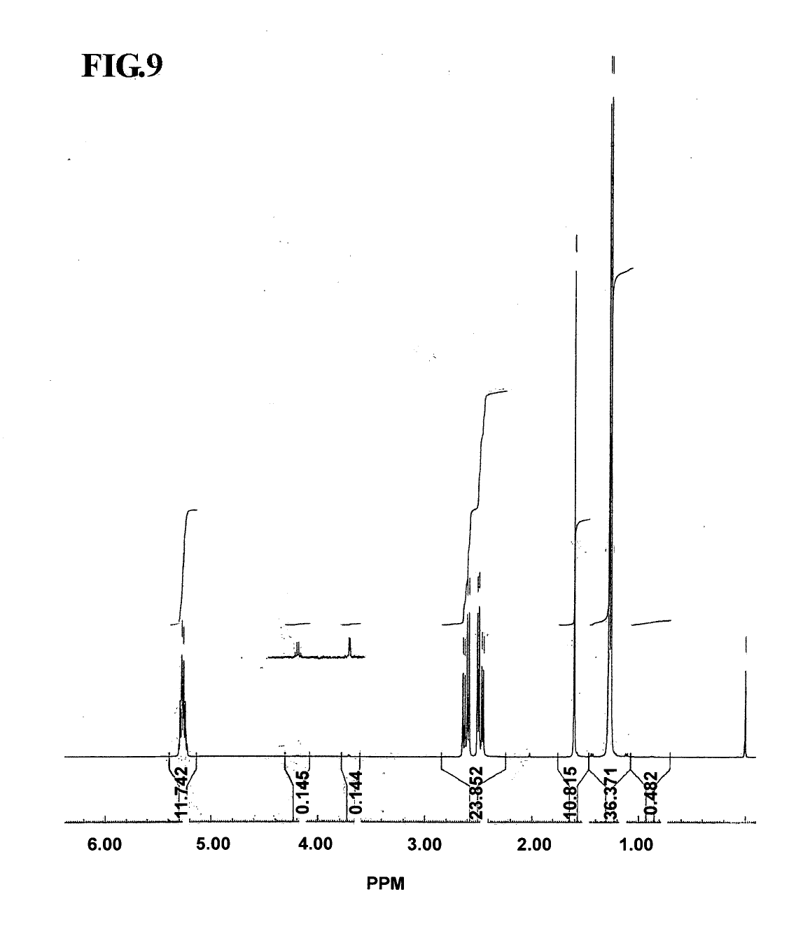

Next, by setting the acetyl-ETG concentration in 500 μL of organic solvent phase reaction solution at 1M and the (R)-3HB concentration in 5 mL of aqueous phase reaction solution at 100 mM, P (3HB) was produced under the same conditions to obtain 6.6 mg product. The structure of the product obtained was confirmed using NMR measurement to find out a P (3HB) product. FIG. 9 shows its ¹H-NMR spectrum.

Then, the molecular weight of the product obtained was measured by gel permeation chromatography (GPC). In GPC measurement method, tandem TSK gel Super HZM-H column (6.0 nmI.D.×150 mm; Tosoh Corporation) was employed and the mobile phase was chloroform with a flow rate of 0.3 mL/min. The calibration curve was determined using pure polystyrene. The weight-average molecular weight (Mw) was 8.5×10⁴, and the molecular weight distribution (Mw/Mn) was 1.7.

While the invention in the document WO2004065609 produced a P (3HB) amount of 1.8 mg, this invention obtained a P (3HB) amount of 6.6 mg, showing approx. 4 times.

From this result, only 2 reaction processes, one process for producing acetyl-thioester from acetate and the other process for synthesizing P (3HB) from acetyl-thioester and released hydroxy acid, can obtain a high-yield of P (3HB) as a final objective substance.

In addition, in the process for producing acetyl-CoA from acetate, acetyl-ETG from ETG can be used to form acetyl-CoA, rather than acetyl-TP from conventional highly toxic TP. Without using a purified enzyme or expensive ATP, acetyl-CoA can be prepared from a low-cost acetate, thereby providing significant advantages in industrial application.

### Example 2

In Example 2, P (3HB) polymerization reaction rate and production amount were discussed according to the type of acetyl-thioester.

As acetyl-thioester, acetyl-TP and acetyl-ETG were employed. In P (3HB) production process, P (3HB) polymerization reaction causes precipitate and makes the reaction solution cloudy. As the polymerization reaction is completed, the reaction solution becomes transparent, with white precipitates. Thus, the progress of P (3HB) production can be found from a visual observation and the turbidity in the reaction solution. The progress of P (3HB) production was measured from the turbidity in the reaction solution using an absorptiometer.

Specifically, 1.5 mL of an organic solvent phase reaction solution, a hexane solution containing 10 mM of acetyl-TP or acetyl-ETG was added to 1.5 mL of an aqueous phase reaction solution containing 100 mM sodium phosphate buffer (pH7.5), 10 mM (R)-3HB, 2.0 mM CoA and 7.5 U (0.3 mg) PCT. Finally, 1.6 U (0.06 mg) PhaC was added to the aqueous phase reaction solution to cause a reaction at 30° C. and the turbidity of the reaction solution was measured at a wavelength of 600 nm using an absorptiometer (Hitachi High-Technologies Corporation).

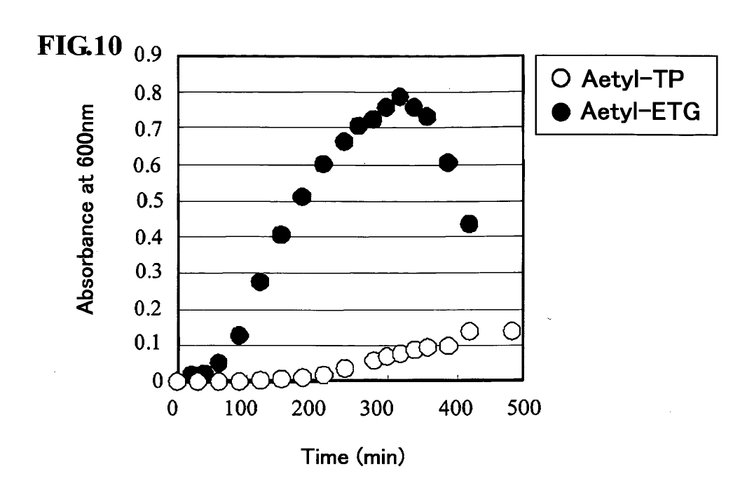

As a result, as shown in FIG. 10, the absorbance of the reaction solution increased in the acetyl-ETG (indicated as in FIG. 10), then decreased after it reached 0.78 in 320 minutes. According to a visual observation, the solution started to become clouded 60 minutes later and in 120 minutes white precipitates were found. Meanwhile, no peak was found in the acetyl-TP (indicated as ∘ in FIG. 10) even 500 minutes later, with an absorbance of the reaction solution at 0.2 or less. Therefore, it was found that the acetyl-ETG provides more rapid reaction and higher-yield product than the acetyl-TP.

It is known that TP is highly toxic due to its blocking activity for PhaC. According to this Example, as opposed to the acetyl-TP, the acetyl-ETG is produced without using TP and also can synthesize P (3HB) suitable for industrial application in an immediate and high-yield manner.

### Example 3

In Example 3, by changing the ratio of the concentration of the acetyl-ETG in the organic solvent phase and the concentration of (R)-3HB in the aqueous phase, the synthetic reaction rate for P (3HB) was discussed.

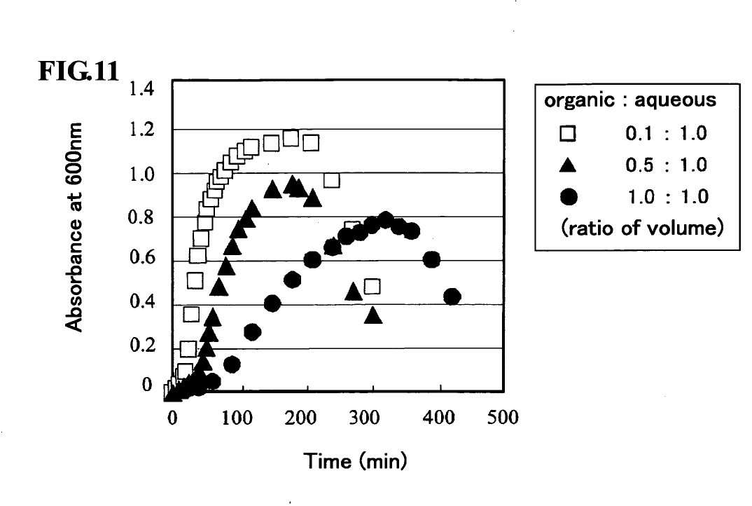

Firstly, a hexane solution containing 0.5 mmol acetyl-ETG was prepared as an organic solvent phase reaction solution, and 1.5 mL of solution containing 0.5 mmol (R)-3HB, 100 mM sodium phosphate buffer (pH7.5), 2.0 mM CoA and 7.5 U (0.3 mg) PCT was prepared as an aqueous phase reaction solution. By maintaining the acetyl-ETG amount in this organic solvent phase reaction solution and the (R)-3HB amount in the aqueous phase reaction solution at constant levels, the amount of the organic solvent phase reaction solution was changed. Specifically, the ratio of the volume of organic solvent phase reaction solution and the volume of the aqueous phase reaction solution was determined at 0.1:1 (indicated as □ in FIG. 11), 0.5:1 (indicated as ▴ in FIG. 11) and 1:1 (indicated as in FIG. 11). Then, 1.6 U (0.06 mg) PhaC was added to the aqueous phase to be reacted at 30° C. The turbidity of the reaction solutions in each system was measured at a wavelength of 600 nm with an absorptiometer.

As a result, as shown in FIG. 11, it was found that the time for reaching the peak in the absorbance and its absorbance are long and low, respectively, as the volumetric ratio of the organic solvent phase grows. In the system in which the ratio of the organic solvent phase and the aqueous phase is 0.1:1 (indicated as □ in FIG. 11), the solution started to become clouded 10 minutes after the reaction started and 60 minutes later, the absorbance reached the peak. Compared with other systems, the time for reaching the peak in the absorbance is the shortest and the absorbance was highest.

From these results, P (3HB) can be synthesized in an immediate and high-yield manner in favorable industrial application when the volumetric ratio of the organic solvent phase and the aqueous phase is 1:1 to 0.1:1, or the ratio (mmol/L) of the concentration of acetyl-ETG and the concentration of (R)-3HB is 1:1 to 10:1, and most preferably when the ratio of the concentration in mol is 10:1.

### Example 4

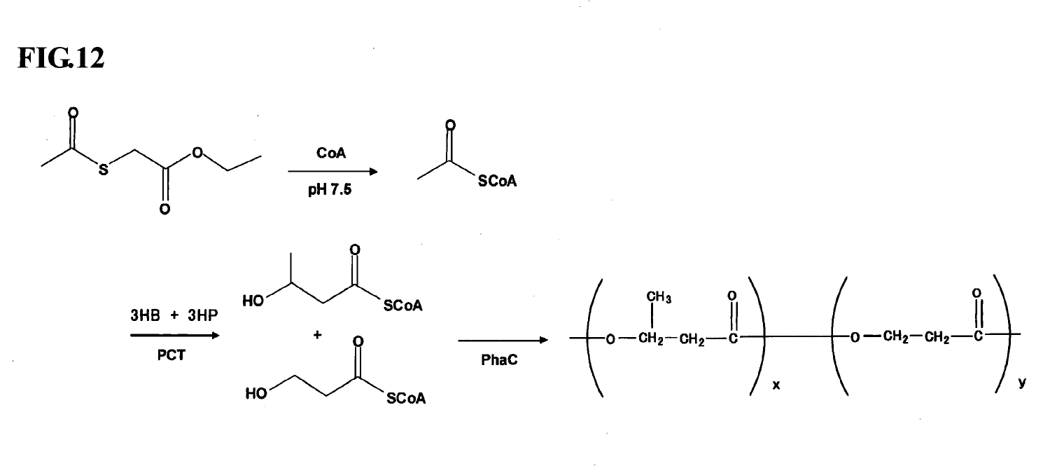

In Example 4, using acetyl-ETG, PhaC and PCT obtained in Examples 1 (1) to (3), P (3HB-co-3HP) was produced. FIG. 12 shows the reaction process.

Firstly, as an aqueous phase reaction solution, 5 mL of a solution containing 100 mM sodium phosphate buffer (pH7.5), and 2.0 mM CoA, and 50 mM 3HP or 50 mM (R)-3HB, and 4.3 U (2.5 mg) PCT was prepared. Next, as an organic solvent phase reaction solution, 500 μL of hexane solution containing 1.0M acetyl-ETG was prepared. Then, after pouring the aqueous phase reaction solution into a screw cap test tube, the organic solvent phase reaction solution was added thereto. Finally, 5 U (2.5 mg) PhaC was added to the aqueous phase to be reacted at 30° C. for 24 hours. After the reaction was completed, the organic solvent phase was removed and 5 mL of chloroform was added thereto, and a product was extracted at 70° C. for 3 hours. The extract was filtrated with a filter (0.2 μm PTFE membrane; Advantec) and 50 mL methanol was added thereto and allowed to stand overnight at 4° C. The resulting precipitate was filtrated with a filter (0.2 μm PTFE membrane) and collected. Vacuum-dried yield was measured to obtain 3.2 mg of product.

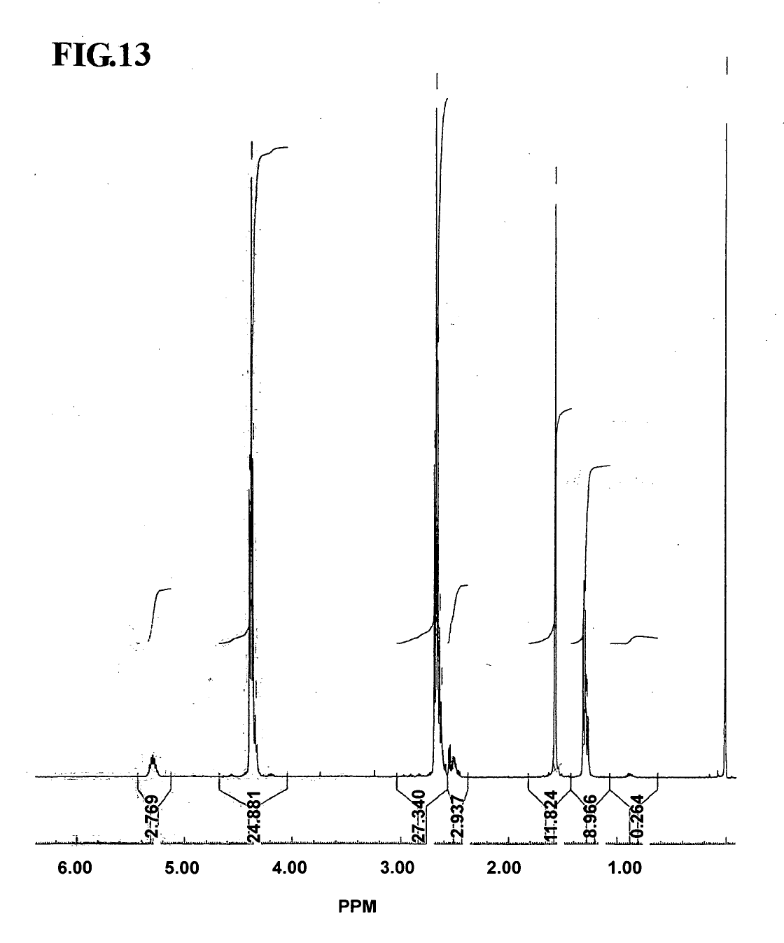

The structure of each product obtained was confirmed in NMR to find out a P (3HB-co-3HP) product. FIG. 13 shows its ¹H-NMR spectrum.

From these results, the method for producing a polymer of this embodiment can produce not only P (3HB), but also P (3HB-co-3HP).

### Example 5

In Example 5, using acetyl-ETG and PCT obtained in Examples 1 (1) to (3) and PhaC obtained as follows, P (3HB-co-LA) was produced. FIG. 14 shows the reaction process.

(1) PhaC

After microbial production of overexpression PhaC was constructed, purified PhaC was obtained (Satoh, Y.; Tajima, K.; Tannai, H.; Munekata, M. J. Biosci. Bioeng. 2003, 95, 335-341).

Firstly, Gene fragment (Sequence No. 1) which codes for Sequence No. 2, the amino-acid sequence of Pseudomonas sp. 61-3-derived PhaC that was disclosed in the document (WO2003-100055), was chemically-synthesized. This gene fragment was inserted into pUC19 treated with restriction enzymes of SacI (TAKARA Bio Inc.) to obtain a plasmid pUC1dm.

Next, a gene fragment having 1.6 kbp of BamHI site and HindIII site amplified by PCR according to the following conditions, using pUC1dm as a template, and vector pQE 30 (Qiagen™) treated with BamHI and HindIII were mixed to be ligated. Then, using this reaction solution, Escherichia coli JM109 was transformed to obtain a plasmid pQC1dm having the PhaC gene from the transfectant. By transfecting this plasmid to an Escherichia coli BL21, Escherichia coli for preparing PhaC was obtained.

The PCR employed the following primers.

Sense primer: ccggatccagtaacaagaatagcgatgacttga (Sequence No. 5)

Antisense primer: tttaagcttaacgttcatgcacatacgtg (Sequence No. 6)

The PCR was performed in 30 cycles, each cycle comprising 60-second reaction at 94° C., 30-second reaction at 55° C., and 100-second reaction at 72° C.

Escherichia coli for preparing PhaC obtained was cultured in 1000 mL of LB medium containing ampicillin at 30° C. for 3 hours and more cultured in the isopropyl-β-D-thio-galactopyranoside (IPTG; 0.25M final concentration)-added LB medium at 30° C. for 16 hours. After sonication of the microbial cell bodies accumulated PhaC, soluble protein in the microbial cell body was collected. The collected protein was put in an Ni-NTA agarose gel column (Qiagen™) to purify (6×His)-PhaC in one step.

(2) Production of P (3HB-co-LA)

Firstly, as an aqueous phase reaction solution, 5 mL of solution containing 100 mM sodium phosphate buffer (pH7.5), 1.0 mM CoA, 50 mM LA (D-enantiomer of LA), 50 mM (R)-3HB and 50 U (2.5 mg) PCT was prepared. Next, as an organic solvent phase reaction solution, 5 mL of hexane solution containing 100 mM acetyl-ETG was prepared. After pouring the aqueous phase reaction solution into a screw cap test tube, the organic solvent phase reaction solution was added thereto. Finally, 0.05 U (2.5 mg) PhaC was added to the aqueous phase to be reacted at 30° C. for 72 hours. The organic solvent phase was removed after the reaction was completed, and 5 mL of chloroform was added thereto to extract a product at 70° C. for 3 hours. The extract was filtrated with a filter (0.2 μm PTFE membrane; Advantec), and 50 mL of methanol was added thereto and allowed to stand overnight at 4° C. Afterwards, a produced precipitate was filtrated with a filter (0.2 μm PTFE membrane) and collected. After it was vacuum-dried, its yield was measured. 0.1 mg of a product was obtained.

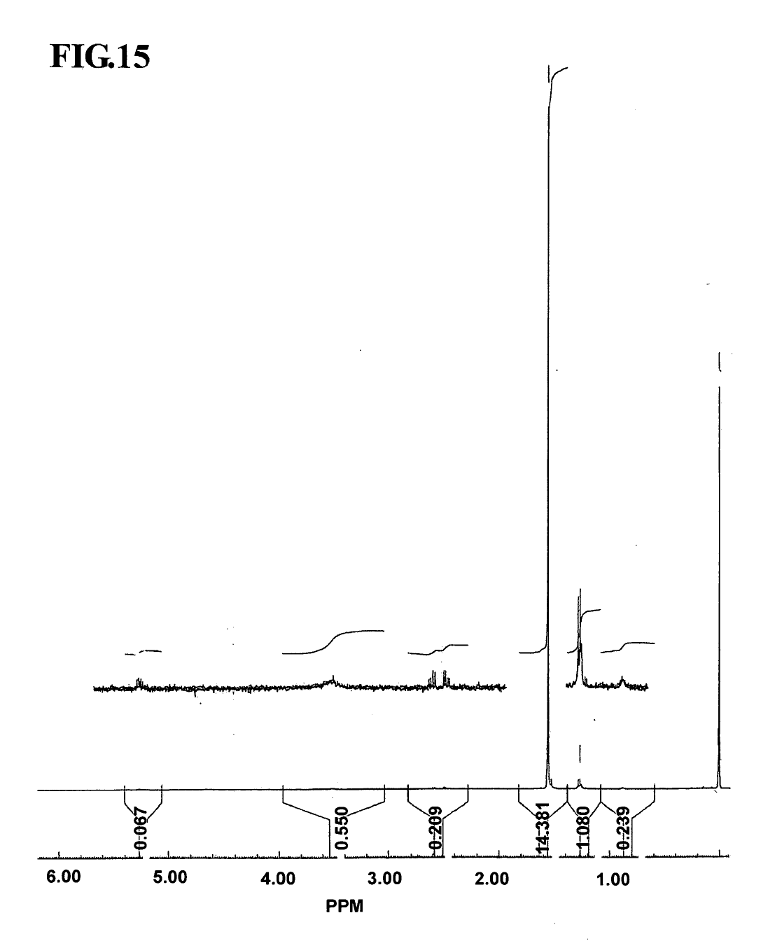

The structure of the product obtained was confirmed using NMR to find out a P (3HB-co-LA) product. FIG. 15 shows this ¹H-NMR spectrum.

From this results, the method for producing a polymer of this embodiment can produce not only P (3HB) or P (3HB-co-3HP), but also P (3HB-co-LA).

### Example 6

In Example 6, PCT substrate specificity was discussed, in order to examine whether the method for producing a polymer in the aqueous-organic solvent two-phase systems performed in the preceding Examples can be applied even in hydroxy acid or unsaturated fatty acid other than (R)-3HB and 3HP. Clostridium propionicum-derived PCT obtained in Example 1 (3) was used.

In the method in Example 1 (3), various types of hydroxy acid or unsaturated fatty acid were added, instead of (R)-3HB, to be reacted for 24 hours with no PhaC added. The compound obtained was analyzed by HPLC (Shimazu). The HPLC measurement employed Mightysil RP-18 GP Aqua-column (4.6 nm I.D.×150 mm; Kanto Chemical), and the mobile phase was liquid A (50 mM NaH₂PO₄ solution containing 10 wt % methanol) or liquid B (50 mM NaH₂PO₄ solution containing 40 wt % methanol). The proportion of the liquid B is 0% (0 to 5 minutes), 0 to 20% (5 to 10 minutes), 20 to 100% (15 to 17.5 minutes), 100% (17.5 to 22.5 minutes), 100 to 0% (22.5 to 25 minutes) or 0% (25 to 30 minutes). The flow rate was 0.7 mL/min, and the detector was an ultraviolet absorptiometer.

As a result, as PCT substrate, or hydroxy acid or unsaturated fatty acid as monomer component, the method can be applied in LA, 3HP, 3HB, 4HB, crotonate, pentenate, serine, glycolate and acrylate.

Clostridium propionicum-derived PCT was used in the Examples, but when other strain-derived PCT is employed, it seems that the method according to the present invention can be applied in hydroxy acid not shown in the Examples. Specifically, by selecting hydroxy acid, or strain of microoganism from which PCT and PHA synthase derives, accordingly, a polymer having a desired monomer composition can be produced.

From the above observations of this embodiment, biodegradable polymers can be produced with the following characteristics, according to the extremely industrially favorable production method.

1. Compared with conventional synthetic methods, this method can produce a polymer in an immediate and high-yield manner.
2. In fact, only two easy reaction processes, one process for forming an acetyl-thioester from acetate and the other process for synthesizing a polymer from acetyl-thioester and released hydroxy acid, can obtain a polymer.
3. An acetyl-CoA can be formed from a low-cost acetate, without using purified enzyme, expensive ATP or highly toxic TP to produce polymers in an immediate and high-yield manner.
4. With wider varieties of monomer components to be selected, a polymer can be produced so as to be provided with a desired composition.

The method for producing a polymer according to the present invention is not limited to the above mentioned embodiment, but may be modified accordingly.

1: A method for producing a polymer, comprising the following reactions:
a) a chemical thioester exchange reaction, wherein an acetyl-thioester is reacted with CoA for forming acetyl-CoA;
b) a monomer-producing reaction, wherein at least one monomer precursor compound is reacted with said acetyl-CoA for forming a (monomer precursor)-CoA derivative; and
c) a polymerization reaction, wherein said (monomer precursor)-CoA derivative is polymerized for forming said polymer comprising units of said monomer.

2: The method for producing a polymer according to claim 1, wherein the monomer precursor compound is a carboxylic acid, preferably a hydroxy acid.

3: The method for producing a polymer according to claim 1, wherein said polymer is a polyester comprising units selected from serine or hydroxyalkanoates, preferably lactate (LA), 3-hydroxypropionate (3HP), 3-hydroxybutyrate (3HB), or 4-hydroxybutyrate (4HB), alone or in combination with each other or with other units.

4: The method for producing a polymer according to claim 3, wherein said polyester is preferably one selected from the group consisting of polylactate (PLA), poly-3-hydroxypropionate {P(3HP)}, poly-3-hydroxybutyrate {P (3HB)}, poly-4-hydroxybutyrate {P(4HB)}, LA-3HP-copolyester {P(LA-co-3HP)}, LA-3HB-copolyester {P(LA-co-3HB)}, LA-4HB-copolyester {P(LA-co-4HB)}, 3HP-3HB-copolyester {P(3HP-co-3HB)}, 3HP-4HB-copolyester {P(3HP-co-4HB)}, and 3HB-4HB-copolyester {P(3HB-co-4HB)}.

5: The method for producing a polymer according to claim 3, is preferably Dextro-rotatory-lactate (D-lactate).

6. The method for producing a polymer according to claim 1, wherein the chemical thioester exchange reaction preferably comprises forming acetyl-CoA from CoA, which is released from the (monomer precursor)-CoA derivative in said polymerization reaction, and said acetyl-thioester.

7: The method for producing a polymer according to claim 1, wherein said acetyl-thioester of the chemical thioester exchange reaction is prepared from an acetate and a thiol compound by a thioesterification reaction.

8: The method for producing a polymer according to claim 1, wherein said thioesterification reaction comprises forming acetyl-thioester from acetate, which is released in said monomer-producing reaction, and thiol compounds.

9: The method for producing a polymer according to claim 7, wherein the thiol compound is ethylthioglycolate (ETG).

10: The method for producing a polymer according to claim 1, wherein an enzyme used for the monomer-producing reaction is one using acetyl-CoA as a substrate.

11: The method for producing a polymer according to claim 1, wherein the chemical thioester exchange reaction, the monomer-producing reaction and the polymerization reaction proceed concurrently in an one pot reaction system, the one pot reaction system preferably comprising an organic solvent phase and an aqueous solvent phase.

12: The method for producing a polymer according to claim 11, wherein the organic solvent phase contains the acetyl-thioester, and wherein the aqueous phase contains CoA, the monomer precursor compound and two enzymes catalyzing said reaction (b) and (c), respectively.

13: The method for producing a polymer according to claim 11, wherein a ratio of concentration of the acetyl-thioester and concentration of the monomer precursor compound is 1:1 to 10:1.

14: A chemo-enzymatically synthesized polymer produced by a method according to any one of claims 1 to 13 and having a molecular weight distribution of the polymer between 1 and 2.5.


SEQUENCE LISTING (6 sequences)

SEQ ID NO: 1 - DNA, 1680 bp, Pseudomonas sp.61-3
atgagtaacaagaatagcgatgacttgaatcgtcaagcctcggaaaacaccttggggcttaaccctgtcatcggcctgcgtggaaaagatctgctgacttctgcccgaatggttttaacccaagccatcaaacaacccattcacagcgtcaagcacgtcgcgcattttggcatcgagctgaagaacgtgatgtttggcaaatcgaagctgcaaccggaaagcgatgaccgtcgtttcaacgaccccgcctggagtcagaacccactctacaaacgttatctacaaacctacctggcgtggcgcaaggaactccacgactggatcggcaacagcaaactgtccgaacaggacatcaatcgcgctcacttcgtgatcaccctgatgaccgaagccatggccccgaccaacagtgcggccaatccggcggcggtcaaacgcttcttcgaaaccggcggtaaaagcctgctcgacggcctcacacatctggccaaggacctggtaaacaacggcggcatgccgagccaggtggacatgggcgctttcgaagtcggcaagagtctggggacgactgaaggtgcagtggttttccgcaacgacgtcctcgaattgatccagtaccggccgaccaccgaacaggtgcatgagcgaccgctgctggtggtcccaccgcagatcaacaagttttatgtgtttgacctgagcccggataaaagcctggcgcgcttctgcctgagcaacaaccagcaaacctttatcgtcagctggcgcaacccgaccaaggcccagcgtgagtggggtctgtcgacttacatcgatgcgctcaaagaagccgtcgacgtagtttccgccatcaccggcagcaaagacatcaacatgctcggcgcctgctccggtggcattacctgcaccgcgctgctgggtcactacgccgctctcggcgagaagaaggtcaatgccctgacccttttggtcaccgtgctcgacaccaccctcgactcccaggttgcactgttcgtcgatgagaaaaccctggaagctgccaagcgtcactcgtatcaggccggcgtgctggaaggccgcgacatggccaaagtcttcgcctggatgcgccctaacgacctgatctggaactactgggtcaacaactacctgctgggtaacgagccaccggtcttcgacattcttttctggaacaacgacaccacccggttgcctgctgcgttccacggcgatctgatcgaaatgttcaaaaataacccactggtgcgcgccaatgcactcgaagtgagcggcacgccgatcgacctcaaacaggtcactgccgacatctactccctggccggcaccaacgatcacatcacgccctggaagtcttgctacaagtcggcgcaactgttcggtggcaaggtcgaattcgtgctgtccagcagtgggcatatcaagagcattctgaacccgccgggcaatccgaaatcacgttacatgaccagcaccgacatgccagccaccgccaacgagtggcaagaaaactcaaccaagcacaccgactcctggtggctgcactggcaggcctggcaggccgagcgctcgggcaaactgaaaaagtccccgaccagcctgggcaacaaggcctatccgtcaggagaagccgcgccgggcacgtatgtgcatgaacgttaa

SEQ ID NO: 2 - protein, 559 aa, Pseudomonas sp.61-3
MSNKNSDDLNRQASENTLGLNPVIGLRGKDLLTSARMVLTQAIKQPIHSVKHVAHFGIELKNVMFGKSKLQPESDDRRFNDPAWSQNPLYKRYLQTYLAWRKELHDWIGNSKLSEQDINRAHFVITLMTEAMAPTNSAANPAAVKRFFETGGKSLLDGLTHLAKDLVNNGGMPSQVDMGAFEVGKSLGTTEGAVVFRNDVLELIQYRPTTEQVHERPLLVVPPQINKFYVFDLSPDKSLARFCLSNNQQTFIVSWRNPTKAQREWGLSTYIDALKEAVDVVSAITGSKDINMLGACSGGITCTALLGHYAALGEKKVNALTLLVTVLDTTLDSQVALFVDEKTLEAAKRHSYQAGVLEGRDMAKVFAWMRPNDLIWNYWVNNYLLGNEPPVFDILFWNNDTTRLPAAFHGDLIEMFKNNPLVRANALEVSGTPIDLKQVTADIYSLAGTNDHITPWKSCYKSAQLFGGKVEFVLSSSGHIKSILNPPGNPKSRYMTSTDMPATANEWQENSTKHTDSWWLHWQAWQAERSGKLKKSPTSLGNKAYPSGEAAPGTYVHER

SEQ ID NO: 3 - DNA, 30 bp, Artificial Sequence
  sense primer
aaggatccatggcgaccggcaaaggcgcgg

SEQ ID NO: 4 - DNA, 24 bp, Artificial Sequence
  antisense primer
tgcagcggaccggtggcctcggcc

SEQ ID NO: 5 - DNA, 33 bp, Artificial Sequence
  sense primer
ccggatccagtaacaagaatagcgatgacttga

SEQ ID NO: 6 - DNA, 29 bp, Artificial Sequence
  antisense primer
tttaagcttaacgttcatgcacatacgtg